(19) United States
(12) Patent Application Publication
(10) Pub. No.: US 2013/0289712 A1
(43) Pub. Date: Oct. 31, 2013

(54) VALVED CONDUIT

(71) Applicant: Shangyi Ji, Shenzhen (CN)

(72) Inventor: Shangyi Ji, Shenzhen (CN)

(21) Appl. No.: 13/860,862

(22) Filed: Apr. 11, 2013

(30) Foreign Application Priority Data
Apr. 26, 2012 (CN) PCT/CN2012/074743

(51) Int. Cl. A61F 2/06

(52) U.S. Cl. CPC A61F 2/06
USPC 623/1.24

(57) ABSTRACT

The present invention discloses a valved conduit, which comprises an artificial blood vessel and an artificial valve. The artificial valve is located at the proximal end of said artificial blood vessel. The lateral wall of the artificial blood vessel is provided with left and right openings at positions close to said artificial valve. The positions of said left and right openings correspond to the ostia of the left and right coronary arteries. The valved conduit also comprises a hard tube which is connected with said artificial blood vessel. The hard tube is bound with the aorta through a ligature and a space is formed between the ligation position and the aortic valve. Thereby it is unnecessary to suture the openings and the coronary arteries and reducing the probability of hemorrhage at the anastomotic site.

### BACKGROUND OF THE INVENTION

1. Technical Field

The present invention relates to the field of valved conduits, in particular to a valved conduit having a hard tube.

2. Description of Related Art

A valved conduit is an artificial blood vessel with a valve. The valved conduit comprises an artificial blood vessel and an artificial valve. The use of the valve conduit simplifies the aortic root operation. The traditional aortic root operation is as follows: cutting the aorta, removing the pathological valve, removing the original arterial blood vessel; directly suturing the proximal end and distal end of the artificial valve to the heart and the artery, directly cutting at the position of the artificial blood vessel corresponding to the coronary artery, and suturing the artificial blood vessel with the coronary artery. This operation causes problems: the position where the artificial blood vessel and the coronary artery are sutured is weak and under a relatively high blood pressure, so the probability of hemorrhage is relatively high. this is the major cause of failure of the aortic root operation and death of patients.

### BRIEF SUMMARY OF THE INVENTION

The technical problem to be solved in the present invention is to provide a novel valved conduit. By using the hard tube provided by the present invention, the original coronary artery can be retained in the aortic root operation, and suturing the artificial blood vessel and the coronary artery can be avoided, thereby reducing the probability of hemorrhage.

To solve the above technical problems, a technical solution adopted by the present invention is as follows:

A valved conduit is provided, which comprises an artificial blood vessel and an artificial valve; said artificial blood vessel has a proximal end and a distal end; and said artificial valve is located at the proximal end of said artificial valve. The lateral wall of said artificial blood vessel is provided with left and right openings at positions close to said artificial valve; the positions of said left and right openings correspond to the ostia of the left and right coronary arteries; said valved conduit also comprises a hard tube which is connected with said artificial blood vessel; said hard tube is bound with the aorta through a ligature, wherein the hard tube comprises a proximal-end hard tube which is located between the left and right openings and said distal end and also is close to the proximal end of said artificial blood vessel.

Wherein, said hard tube also comprises a distal-end hard tube arranged at the distal end of said artificial blood vessel.

Wherein, the outer surface of said hard tube is provided with at least one ring slot along the circumference; and said ring slot is used for fixing said ligature.

Wherein, the distances between said left and right openings and said artificial valve are 2-3 cm.

Wherein, the distances between one end of said proximal-end hard tube close to said proximal end and the said left and right openings are 1-2 cm.

Wherein, the material of said hard tube may be metal, carbon, or plastic.

Wherein, said metal may be stainless steel, titanium alloy or other metal.

Wherein, the two ends of said hard tube are respectively sutured with the cuts of the sheared artificial blood vessel.

Wherein, said hard tube is sleeved in the cavity of said artificial blood vessel.

Wherein, the sizes of said left and right openings are 2-3 mm; and the edges of said left and right openings are processed by carbon, metal, organic material, or ceramic.

The present invention has the following benefits: by using the valved conduit provided by the present invention, the coronary artery is retained in the operation; only the positions on the artificial blood vessel corresponding to the ostia of the left and right coronary arteries are respectively provided with a 2-3 mm opening; a proximal-end hard tube is located between the opening and the distal end and is 1-2 cm away from the opening; in addition, the proximal-end hard tube and the aorta are bound with a ligature on the outside of the aorta, and then a space is formed between the ligation position and the aorta after ligation; thus, blood flows not only through the whole body along the artificial blood vessel, but also into the space to enter the left and right coronary arteries, and it is unnecessary to suture the openings and the coronary arteries, thereby reducing the probability of hemorrhage at the anastomotic site at the proximal end. Furthermore, in the present invention, the distal end of the artificial blood vessel is also provided with a distal-end hard tube, and the distal-end hard tube is bound and sutured with the aorta by ligation instead of suturing the artificial blood vessel and the aorta, thereby reducing probability of hemorrhage at the anastomotic site of the distal end.

### BRIEF DESCRIPTION OF THE SEVERAL VIEWS OF THE DRAWINGS

FIG. 1 is a schematic view of a valved conduit in the placed state during operation in one embodiment of the present invention.

### DESCRIPTION OF THE SIGNS OF THE MAJOR COMPONENTS

100, valved conduit; 10, artificial blood vessel; 11, left opening; 12, right opening; 200, aorta; 210, ostium of the left coronary artery; 220, ostium of the right coronary artery; 230, space; 20, proximal-end hard tube; 30, ligature; 40, distal-end hard tube.

### DETAILED DESCRIPTION OF THE INVENTION

The technical contents, structural features, objectives and effects of the present invention are described in detail accompanying the following embodiments and attached drawings.

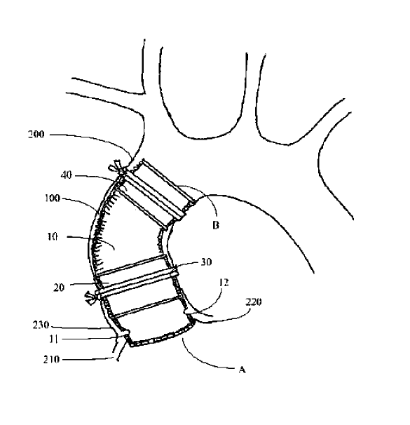

Referring to FIG. 1, a schematic view of the valved conduit 100 in one embodiment of the present invention is provided. A valved conduit 100 comprises an artificial blood vessel 10 and an artificial valve; the artificial blood vessel 10 has a proximal end A and a distal end B; the proximal end A is fitted with the heart, and the distal end B is fitted with the aorta 200. The artificial valve is located at the proximal end A of the artificial blood vessel 10.

The lateral wall of the artificial blood vessel 10 is provided with a left opening 11 and a right opening 12 at positions close to the artificial valve at the proximal end A. The positions of the left opening 11 and the right opening 12 respectively correspond to the ostium of the left coronary artery 210 and the ostium of the right coronary artery 220. In the present embodiment, the axial distances between the left and right openings and the artificial valve along the artificial blood vessel 10 are 2-3 cm, and the sizes of the left and right openings are 2-3 mm. In the present embodiment, the openings are round. In other embodiments, the openings may also be oval, microscler, square, rectangular, triangular, or irregularly shaped. In addition, to avoid thrombopoiesis, the edges of the left and right openings are processed with carbon, metal, organic material, or ceramic. Preferably, the metal is silver and titanium, while the organic material is polytetrafluoroethylene plastic.

The valved conduit 100 also comprises a hard tube which is connected with said artificial blood vessel 10. The hard tube is bound with the aorta through a ligature. Wherein, the material of the hard tube may be metal, carbon, or plastic. Preferably, the metal material is stainless steel or titanium alloy. In the present embodiment, the hard tube is connected with the artificial blood vessel 10 by means of being sleeved in the cavity of the artificial blood vessel 10. In other embodiments, the hard tube is connected with the artificial blood vessel 10 by shearing the artificial blood vessel 10 and then suturing the cuts of the artificial blood vessel 10 with two ends of the hard tube.

The hard tube comprises a proximal-end hard tube 20. The proximal-end hard tube 20 is located between said left and right openings and said distal end B and close to the proximal end A of said artificial blood vessel. In the present embodiment, the axial distances between one end of said proximal-end hard tube and said left and right openings along the artificial blood vessel 10 are 1-2 cm.

The proximal end A of the artificial blood vessel 10 is sutured and connected with the heart. The outside of the aorta 200 at a height corresponding to the distal-end hard tube 20 is provided with the ligature 30. The ligature 30 binds the aorta 200 and the distal-end hard tube 20. In the present embodiment, the hard tube also comprises a ring slot 31 arranged along the circumference of the hard tube. The ring slot 31 is used for fixing said ligature 30. Wherein, the material of the ligature 30 may be plastic, thermoplastic polyurethane elastic plastic, metal, terylene, or an organic material. After ligation, a space 230 is formed at the ligation position and the aorta 200. Blood flows not only through the whole body along the artificial blood vessel 10, but also into the space 230 through the left opening 11 and the right opening 12 to enter the left and right coronary arteries. In this way, suturing the artificial blood vessel and the left and right coronary arteries with a needle and a thread can be avoided, thereby reducing the probability of hemorrhage at the anastomotic site of the proximal end.

The distal end B of the artificial blood vessel 10 and the aorta 200 can be directly sutured or connected and fitted by a lantern ring. In the present embodiment, the valved tube 100 also comprises a distal-end hard tube 40 which is located at the distal end B of the artificial blood vessel 10, wherein the material and aperture of the distal-end hard tube 40 can be identical with or different from those of the proximal-end hard tube 20 according to demands. The outside of the aorta 200 at a height corresponding to the distal-end hard tube 40 is provided with a ligature. The aorta 200 and the distal-end hard tube 40 are bound with the ligature instead of suturing the distal end of the artificial blood vessel and the aorta using the needle and thread, thereby reducing the probability of hemorrhage at the anastomotic site of the distal end.

The valved conduit of the present invention is applicable to all kinds of diseases needing Bentall, Cabrol, and Wheat operations, and all types of aortic root operation s for replacement of the aortic valve. The key points of the operation include:

(1) The method for replacing the aortic valve is identical with Bentall; (2) instead of coronary artery graft, the artificial blood vessel is provided with 2-3 mm openings at positions corresponding to the ostia of the left and right coronary arteries; (3) a hard tube with a supporting force is arranged at a position 1-2 cm higher than the openings, and the hard tube and the aorta are bound using the ligature from the outside of the aorta; after ligation, a space is formed between the ligation position and the aortic valve; then, blood flows not only through the whole body along the artificial blood vessel, but also into the space from said openings to enter the left and right coronary arteries.

Wherein, the distal end of the artificial blood vessel can be sutured with the needle and thread, or another hard tube can be placed and bound with the aorta using the ligature from the outside of the aorta; and then, when necessary, the blood circulation can be temporarily paused within 5 minutes. The hard tube cannot block the ostium of the innominate artery to avoid affecting brain blood supply; after the operation, anticoagulation must be performed in time, and preferably, INR is controlled to be 1.5-2.5 to reduce or avoid thrombopoiesis.

By using the valved conduit provided by the present invention, the coronary artery is retained in the operation; only the positions on the artificial blood vessel corresponding to the ostia of left and right coronary arteries are respectively provided with a 2-3 mm opening; a proximal-end hard tube is located between the opening and the distal end and is 1-2 cm away from the opening; in addition, the proximal-end hard tube and the aorta are bound with a ligature on the outside of the aorta, and then a space is formed between the position of ligation and the aorta after ligation; thus, blood flows not only through the whole body along the artificial blood vessel, but also into the space to enter the left and right coronary arteries, and it is unnecessary to suture the openings and the coronary arteries, thereby reducing the probability of hemorrhage at the anastomotic site at the proximal end. Furthermore, in the present invention, the distal end of the artificial blood vessel is also provided with a distal-end hard tube, and the distal-end hard tube is bound and sutured with the aorta by ligation instead of suturing the artificial blood vessel and the aorta, thereby reducing probability of hemorrhage at the anastomotic site of the distal end.

The above mentioned description is only the exemplary embodiment of the present invention, which cannot limit the present invention. Within the spirit and principle of the present invention, any modification of the equivalent structure or equivalent flow, or direct or indirect application in other related technical fields all shall be included in the protection scope of the present invention.

What is claimed:

1. A valved conduit, comprising an artificial blood vessel and an artificial valve, said artificial blood vessel having a proximal end and a distal end, said artificial valve being located at the proximal end of said artificial valve, characterized in that:
the lateral wall of said artificial blood vessel is provided with left and right openings at positions close to said artificial valve; the positions of said left and right openings correspond to the ostia of the left and right coronary arteries; said valved conduit also comprises a hard tube which is connected with said artificial blood vessel; said hard tube is bound with the aorta through a ligature, wherein the hard tube comprises a proximal-end hard tube which is located between the left and right openings and said distal end and is also close to the proximal end of said artificial blood vessel.

2. The valved conduit according to claim 1, characterized in that:
said hard tube also comprises a distal-end hard tube arranged at the distal end of said artificial blood vessel.

3. The valved conduit according to claim 1, characterized in that:
the outer surface of said hard tube is provided with at least one ring slot along the circumference; and said ring slot is used for fixing said ligature.

4. The valved conduit according to claim 1, characterized in that:
the distances between said left and right openings and said artificial valve are 2-3 cm.

5. The valved conduit according to claim 1, characterized in that:
the distances between one end of said proximal-end hard tube close to said proximal end and the said left and right openings are 1-2 cm.

6. The valved conduit according to claim 1, characterized in that:
the material of said hard tube may be metal, carbon, or plastic.

7. The valved conduit according to claim 6, characterized in that:
said metal may be stainless steel or titanium alloy.

8. The valved conduit according to claim 1, characterized in that:
the two ends of said hard tube are respectively sutured with the cuts of the sheared artificial blood vessel.

9. The valved conduit according to claim 1, characterized in that:
said hard tube is sleeved in the cavity of said artificial blood vessel.

10. The valved conduit according to claim 1, characterized in that:
the sizes of said left and right openings are 2-3 mm; and the edges of said left and right openings are processed by carbon, metal, organic material, or ceramic.
